(19) United States
(12) Patent Application Publication
(10) Pub. No.: US 2006/0183981 A1
(43) Pub. Date: Aug. 17, 2006

(54) Knowledge determination system

(76) Inventor: James E. Skinner, Bangor, PA (US)

Correspondence Address: NEEDLE & ROSENBERG, P.C., SUITE 1000, 999 PEACHTREE STREET, ATLANTA, GA, 30309-3915, US

(21) Appl. No.: 11/332,066

(22) Filed: Jan. 13, 2006

Related U.S. Application Data
(60) Provisional application No. 60/644,440, filed on Jan. 14, 2005.

(51) Int. Cl. A61B 5/00

(52) U.S. Cl. 600/301

(57) ABSTRACT

A method for determining whether a subject has knowledge of a stimulus is described. The method includes generating a sensory signal corresponding to the stimulus for receipt by the subject, and collecting a cerebral indicator signal involuntarily generated in response to the subject processing the sensory signal. Identifying whether degrees of freedom in the cerebral indicator signal of the subject either increased or decreased is also completed. It is determined whether the subject has knowledge of the stimulus depending on whether the degrees of freedom increased or decreased. In addition, the method associates knowledge of the stimulus with the subject if it is determined that the subject has knowledge of the stimulus.

### CROSS REFERENCE TO RELATED APPLICATIONS

This application claims priority to a U.S. Provisional Patent Application with application No. 60/644,440 entitled “KNOWLEDGE DETERMINATION SYSTEM,” which was filed on Jan. 14, 2005. This application is hereby incorporated by reference in its entirety.

### DESCRIPTION OF THE RELATED ART

With the growing quest for more effective modes of communication, determining whether an individual is communicating honestly regarding knowledge on a particular topic may be helpful. For example, conventional medical diagnostic methods depend on whether a patient was being completely truthful about his condition. In addition, law enforcements officials are also quite concerned about whether an individual is communicating honestly about a particular fact. Conventional methods of assessing an individual's knowledge have varied from simply intuition to complex lie-detector tests. While these methods vary in the information used in making the determination, they remain susceptible to both an individual's desire to be dishonest and an inability to communicate. Consequently, there remains an unmet need relating to knowledge determination.

### BRIEF DESCRIPTION OF THE DRAWINGS

FIG. 1A is a block diagram of a knowledge determination system 100 according to the present invention.

FIG. 1B, this figure is an example of a physiological diagram of a simplistic model of a brain of the subject of FIG. 1 illustrating how cerebral indicator signals may be generated after the knowledge determination system 100 processes the sensory signals.

FIG. 1C is a physiological diagram of a human brain that operates like the model of FIG. 1B, and is operated within the known properties of real neurons in a vertebrate brain.

FIG. 1D is a block diagram illustrating an alternative implementation for the knowledge determination system of FIG. 1B when the processor is a computer.

FIG. 2 is a flow chart illustrating a knowledge determination algorithm that controls the knowledge determination software of FIG. 1.

FIG. 3 is a flow chart illustrating the event related potential algorithm of FIG. 2.

FIG. 4 is a flow chart illustrating the PD2i algorithm of FIG. 2.

While the invention is susceptible to various modifications and alternative forms, specific embodiments have been shown by way of example in the drawings and subsequently are described in detail. It should be understood, however, that the description herein of specific embodiments is not intended to limit the invention to the particular forms disclosed. In contrast, the intention is to cover all modifications, equivalents, and alternatives falling within the spirit and scope of the invention as defined by the appended claims.

### DETAILED DESCRIPTION OF EMBODIMENTS

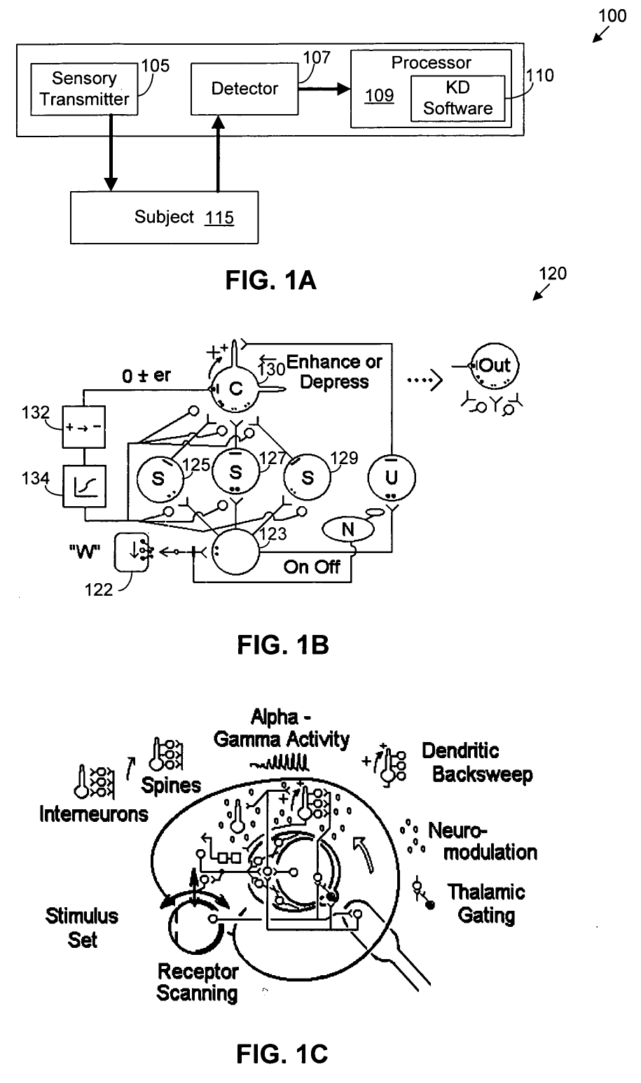

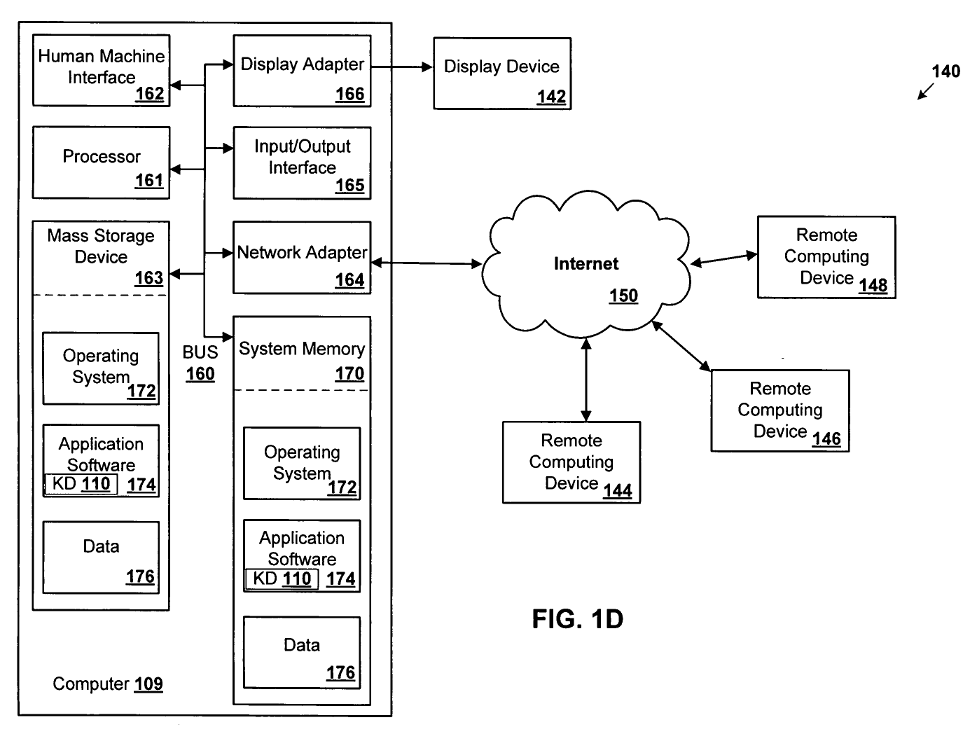

Turning now to the figures, FIG. 1A is a block diagram of a knowledge determination system 100 that includes a sensory transmitter 105, detector 107, and processor 109 according to the present invention. The sensory transmitter 105 may produce any kind of sensory signal that a subject 115 may receive. For example, the sensory transmitter 105 may transmit audible signals, such as naming a particular organization that the subject 115 may hear. Alternatively, the sensory transmitter 105 may transmit a visual signal, such as an image of a location that the subject 115 may see. In another alternative embodiment, the sensory transmitter 105 may use touch as a means of transmitting information. For example, the sensory transmitter 105 may include a Braille display board with certain information entered on the board, such as the name of an organization.

When the subject 115 receives the sensory signals from the sensory transmitter 105, the subject's brain subconsciously and involuntarily transmits cerebral indicator signals after processing the sensory signals. The subject 115 may be a human of any age (e.g., child, adolescent, or adult) so long as the subject's brain is sufficiently developed enough to have learned information. After processing the sensory signals, the subject's brain produces cerebral indicator signals that the detector 107 receives.

Turning now to FIG. 1B, this figure is a physiological diagram of a simplistic model 120 of a brain illustrating how cerebral indicator signals are generated after the knowledge determination system 100 processes the sensory signals. One skilled in the art will realize that this model is both applicable for humans and non humans. The model 120 is a parallel and distributed 1-layer network with seven neurons, though other models may also be used with the knowledge determination system 100. The blank neuron 123 receives an input number n (e.g., 0, 001, 002, . . . 999, 1) that is selected from the world (W) of possibilities by the scanning mechanism 122. The input is placed in the input cell 123 with two dots, to represent post-synaptic effects of placing the number (n). However, the input is only placed in the input cell 123 only if the non-specific cell (N) allows it. This same N cell also allows the input number to pass into the Unspecific (U) neuron.

The model 120 modifies inter-neurons to satisfy timing and gain constraints, which correspondingly produces the cerebral indicator signals. The input cell 123 distributes the input number to the three specific-sensory inter-neurons S, or hidden units, that are labeled 125, 127, and 129. The inter-neurons 125, 127, and 129 each have different synaptic gains (multipliers). When the input number n passes through each of these inter-neurons, the output becomes xn where x is a multiple of n. The collector cell C labeled 130 receives the outputs from the inter-neurons 125, 127, and 129 and sums them. This C cell then modifies the resultant according to the time constraints used with long-term potentiation (LTP) and long-term depression (LTD) from the U cell (large+or small+). LTP and LTD are a synaptic gain effects that result from the intensity and time-dependent flow of information through the U-cell. This cell monitors the flow of sensory input and changes the timing of its output to the C-cell such that other synapses on the C-cell are up or down regulated in gain. To satisfy these timing constraints, the inter-neurons 125, 127, 129 can be made into spines on the dendrites of a real neuron. Making these inter-neurons into spines on the dendrites relates to how a timing input from the unspecific cell U is realized and how this cell interacts with the dendritic backsweep from a successfully activated state in which an action potential relationship develops (see FIG. 1C). The sign for the resulting modified sum in the collector cell 130 is then determined at box 132; this cell uses either a comparison with a “tutored” value (i.e., in a “tutored” neural network) or comparison with the tiring with the U output (i.e., an “untutored” neural network) to determine the sign, which can either increase (+) or decrease (−) the collected value from C. The model 120 then passes the output of box 132 through a nonlinear function (see box 134), such as a sigmoid curve, or any other suitable nonlinear function. The output of box 134 is then sent back to the inter-neurons 125, 127, and 129 in incremental steps, which modifies the corresponding synaptic gains, up or down. Reviewing the outputs of these inter-neurons, as a function of the iterations, illustrates that at least one inter-neuron behaves similar to gamma activity that occurs in a real brain. Because the inter-neurons are intrinsic to the model 120, the resulting gamma-like activity is intrinsic to the knowledge determination system 100.

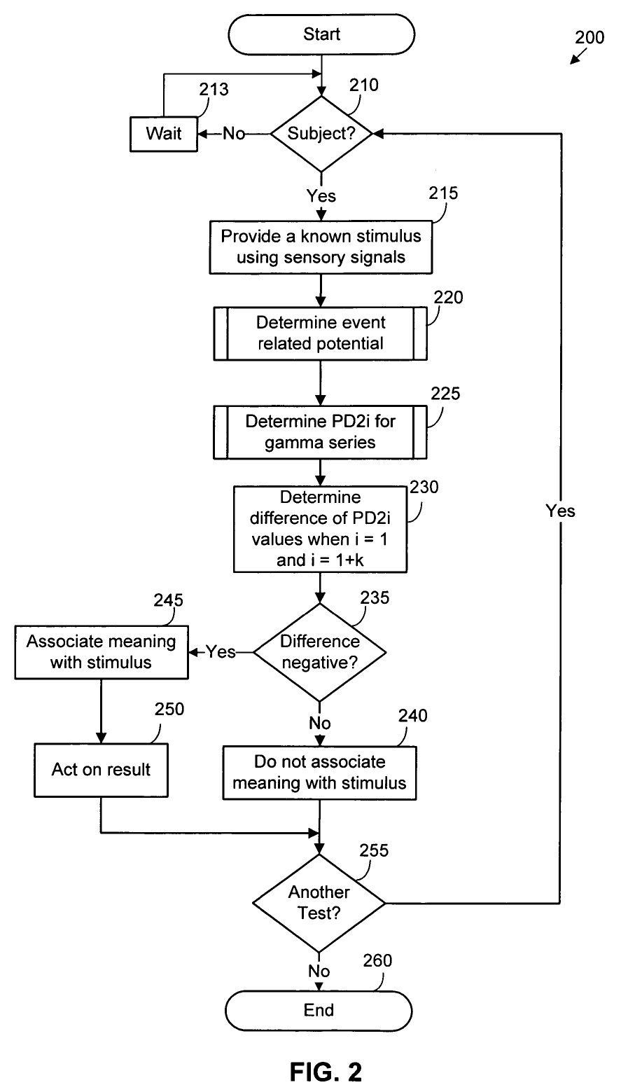

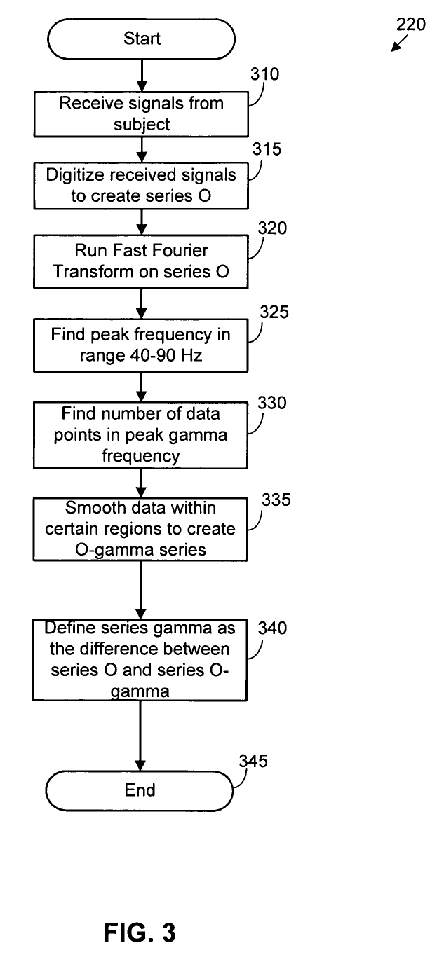

Returning to FIG. 1A, the subject 115 reflexively produces cerebral indicator signals when an input is received, such as a sensory signal. These cerebral indicator signals result from gamma-like activity produced by inter-neurons within the subject's brain as described with reference to FIG. 1C. The knowledge determination system 100 includes the detector 107 positioned to receive the cerebral indicator signals from the subject 115 (e.g., gamma activity as represented from the model illustrated in FIG. 1B or as represented by its placement in a vertebrate brain known to generate such activity (see FIG. 1C)). The detector 107 measure an event related potential of the cerebral indicator signals (e.g., which includes the gamma activity), which is described with reference to FIGS. 2-3. The detector 107 may be a magneto encephalograph (MEG), an electroencephalogram (EEG), or some other suitable device. The detector 107 connects to the processor 109, which receives the detected cerebral indicator signals.

The processor 109 may be any type of conventional processing device, such as a computing system, a microprocessor, or some other suitable device. There may be various types of software within the processor that controls its operation, such as knowledge determination software 110. In an alternative embodiment, the KD software 110 may be hardware, firmware, or some other type of programming logic.

FIG. 1D is a block diagram illustrating an alternative implementation for the knowledge determination system 100 when the processor 109 is a computer. This implementation is only an example and is not intended to suggest any limitation as to the scope of use or functionality of the architecture. Neither should this implementation be interpreted as having any dependency or requirement relating to any one or combination of illustrated components.

The system memory 170 within the computer 109 can be operational with numerous other general-purpose or special purpose computing system environments or configurations. Thus, an environment 140 can be any one of several well known computing environments, such as personal computers, server computers, laptop devices, and multiprocessor systems. Additional examples include set top boxes, programmable consumer electronics (e.g., personal digital assistants), network PCs, minicomputers, mainframe computers, distributed computing environments that include any of the above systems or devices, and the like.

The environment 140 includes several electronic devices including a general-purpose computing device in the form of a computer 109 that houses the system memory 170. To interface with a user (not shown), the computer 109 is connected to a display device 109. In addition, the computer 109 can operate in a networked environment using logical connections to one or more remote computing devices 144-148 by using the Internet 150. These remote computing devices can be located at several different physical locations.

The display device 142 can be one of several types of display devices. For example, the display device 142 can be a CRT (cathode ray tube) display, an LCD (Liquid Crystal Display), or some other suitable type of display. In addition to the display device 142, the computer 109 can connect to other output peripheral devices, such as speakers (not shown), a printer (not shown), and the like.

A user can enter commands and information into the computer 109 via one or more input devices (not shown). The input devices can include, but are not limited to, a keyboard, pointing device (e.g., a “mouse”), a microphone, a joystick, a serial port, a scanner, and the like. These and other input devices can connect to the microprocessor 161 via the human machine interface 162, which is coupled to the system bus 160. Alternatively, this human machine interface may be connected by other interface and bus structures, such as a parallel port, game port, or a universal serial bus (USB).

Like the computer 109, the remote computing devices 140-148 can be a personal computer, portable computer, a server, a router, a network computer, a peer device, or some other suitable device. Logical connections between the computer 109 and the remote computing devices 140-148 can be made via a local area network (LAN) and a general wide area network (WAN). These networks can be wired networks, wireless networks, or the like, such as networks in offices, enterprise-wide computer networks, intranets, or on the Internet 115.

The computer 109 can include numerous components in addition to the system memory 170. For example, the computer 109 can include the system bus 160 that couples various system components to the system memory 170. Other system components can include one or more processors or processing units 161, a human machine interface 162, a mass storage device 163, a network adapter 164, input/output interface 165, and display adapter 166.

The system bus 160 represents one or more of several possible types of bus structures, including a memory bus or memory controller, a peripheral bus, an accelerated graphics port, and a processor or local bus using any of a variety of bus architectures. The architectures can include, for example, an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. The system bus 160 and all buses specified in this description can also be implemented over a wired or wireless network connection. Consequently, the remote devices 140-148 can include components, such as mentioned above, connected by the system bus 160, which in effect implements a distributed computing system.

In addition, the computer 109 can include a variety of accessible computer readable media. For example, this media can include volatile media, non-volatile media, removable and non-removable media depending on the type of system component that the media is used within. For example, the mass storage device 163 can use non-volatile media for storing computer code, computer readable instructions, data structures, program modules, and other data for the computer 109. Consequently, the mass storage device 163 can be a hard disk, a removable magnetic disk, a removable optical disk, magnetic cassettes or other magnetic storage devices, flash memory cards, CD-ROM, digital versatile disks PVD) or other optical storage, random access memories (RAM), read only memories (ROM), electrically erasable programmable read-only memory (EEPROM), and the like.

For purposes of illustration, application programs and other executable program components such as the operating system 172 are illustrated herein as discrete blocks. However, it is recognized that such programs and components reside at various times in different storage components of the computing device 109, and are executed by the data processor(s) of the computer 109. An implementation of application software 174 may be stored on or transmitted across some form of computer readable media. Computer readable media can be any available media that can be accessed by a computer. By way of example, and not limitation, computer readable media may comprise “computer storage media” and “communications media.” “Computer storage media” can include volatile and non-volatile, removable and non-removable media implemented in any method or technology for storage of information such as computer readable instructions, data structures, program modules, or other data. Computer storage media includes, but is not limited to, random access memory (RAM), read only memory (ROM), electrically erasable programmable ROM (EEPROM), flash memory or other memory technology, CD-ROM, digital versatile disks PVD) or other optical storage, magnetic cassettes, magnetic tape, magnetic disk storage or other magnetic storage devices, or any other medium which can be used to store the desired information and is accessible by the computer 109.

Any number of program modules can be stored on the mass storage device 163, including by way of example, an operating system 172 and application software 174. Each of the operating system 172 and application software 174 (or some combination thereof) may include elements of the programming and the application software 174. More specifically, the application software 174 can include the knowledge determination software 110 of FIG. 1 that is described with reference to other figures. Data 176 can also be stored on the mass storage device 163. Data 176 can be stored in any of one or more databases known in the art. Examples of such databases include, DB2®, Microsoft® Access, Microsoft® SQL Server, Oracle®, mySQL, PostgreSQL, and the like. These databases can be centralized or distributed across multiple systems.

The system memory 170 can include computer readable media in the form of volatile memory, such as random access memory (RAM), and/or non-volatile memory, such as read only memory (ROM). The system memory 170 typically contains data such as data 176 and and/or program modules such as operating system 172 and application software 176 that are immediately accessible to and/or are presently operated on by the microprocessor 161.

Turning now to FIG. 2, this figure is a flow chart illustrating a knowledge determination (KD) algorithm 200 that controls the knowledge determination software 110. The knowledge determination algorithm 200 begins at block 210. In this step, the KD algorithm 200 determines whether a subject has been received. When a subject has been received, a cerebral indicator signal is present at the detector 107. By assessing whether an cerebral indicator signal is present at the detector 107, it is possible to determine whether a subject has been received. If a subject has not been received, the “no” branch is followed from step 210 to step 213. In step 213, the KD algorithm 200 waits a preselected period, such as 3 seconds, 15 minutes, 60 minutes, or some other suitable period. After step 213, the KD algorithm 200 repeats step 210. In other words, the loop repeats until a subject has been received or the algorithm ends because of a time out. When it is determined that the subject 115 was received, the “yes” branch is followed from step 210 to step 215. In step 215, the KD algorithm 200 provides a suspected known stimulus using sensory signals, such as audible, visual, or touch signals. For example, if the subject could potentially know the name of a gangster, then the test, or suspected known, stimulus would be an auditory stimulus, saying the gangster's name.

Step 215 is followed by step 220. In this step, the KD algorithm 200 determines the event related potential for the cerebral indicator signals using the event related potential subroutine. The event related potential (ERP) is essentially a computer average of the potential for one to several trials (e.g., 1, 4, 5, or some other suitable number), where the same stimulus is presented. For the knowledge determination system 100, the degrees of freedom for the ERP is determined using the ERP subroutine 220, which is described in greater detail with reference to FIG. 3.

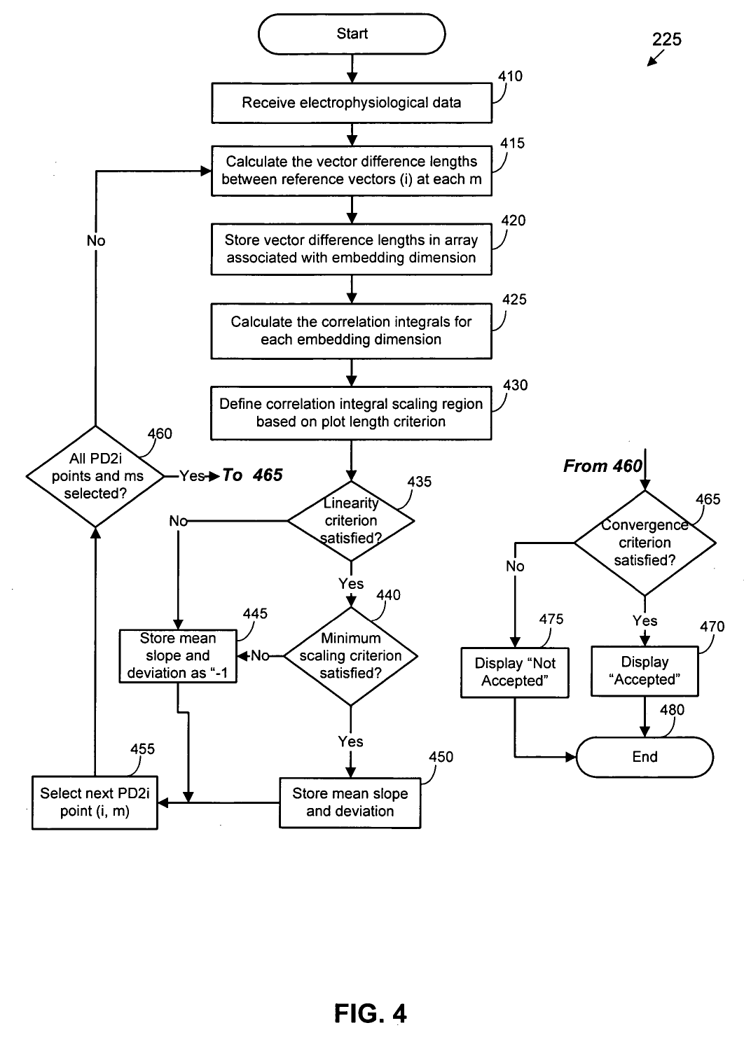

Step 220 is followed by step 225. In this step the KD algorithm 200 determines a PD2i by running the PD2i subroutine for a gamma series associated with the gamma like activity described in FIG. 1B. The PD2i subroutine was described in detail in U.S. Pat. No. 5,709,214 entitled PD2I Electrophysiological Analyzer issued to James E. Skinner on Jan. 20, 1998 and U.S. Pat. No. 5,720,294 entitled PD2I Electrophysiological Analyzer issued to James E. Skinner on Feb. 24, 1998, which are hereby incorporated by reference. Essentially, the PD2i subroutine 225 is a nonlinear-deterministic mathematical model that measures the degrees of freedom of data from the subject 115. The knowledge determination algorithm 200 determines whether the degrees of freedom within the knowledge determination system 100 either increased or decreased (see steps 230, 235). In other words, the PD2i subroutine calculates the nonlinear degrees of freedom of the enriched biological EEG data (i.e., the cerebral indicator signals received from the subject 115 and captured by the detector 107). The PD2i subroutine 225 is described in greater detail with reference to FIG. 4.

Step 225 is followed by step 230, which evaluates the nonlinear degrees of freedom result. In step 230, the KD algorithm 200 determines the difference in the PD2i values for the situations where i=1 and i=1+k where i is the PD2i value at the first point of a PD2i series just after the stimulus and k is a subsequent point, after a delay of 200 ms, by which the brain has analyzed the stimulus fully. Other possible delay values can include 100 ms, 250 ms, or some other suitable delay. Step 230 is followed by step 235 where the KD algorithm 200 determines whether the PD2i=1−PD2i=k is negative. If the difference is not negative, the “no” branch is followed from step 235 to step 240. In step 240, the KD algorithm 200 does not report an association of meaning with the stimulus. In other words, this step determines that the subject 115 did not have prior learned knowledge (meaning) associated with the stimulus.

If it is determined at step 235 that the difference is negative, the “yes” branch is followed from step 235 to step 245. In this step, the KD algorithm 200 reports that meaning is associated with the stimulus. In other words, the KD algorithm 200 notes that the subject did have previous knowledge of the stimulus. Step 245 is followed by step 250. In this step, the KD algorithm 200 acts on the result. For example, the KD algorithm 200 may send notice to an operator that the subject should be detained for further questioning. Thus, the KD algorithm can use the PD2i and detect when there are gamma activity shifts based on local or global synchronization and associate meaning with the presented stimulus. Additional details regarding local versus global gamma synchronization can be found in the article entitled “Event related dimensional reduction in the primary auditory cortex of the conscious cat are revealed by new techniques for enhancing the nonlinear dimensional algorithms,” written by James Skinner and Mark Molnar, which published in the International Journal of Psychophysiology in 1999. Both step 240 and step 250 are followed by step 255. In step 255, the KD algorithm 200 determines whether there is another trial that should be completed. Another test trial can be done if there was a malfunction in the previous calculation or extraneous noise in the defected signal. If another trial needs to be completed, the “yes” branch is followed from step 255 back to step 210 and the KD algorithm 200 repeats. Otherwise, the “no” branch is followed from step 255 to step 260. In step 260, the KD algorithm 200 ends because it has determined whether the subject 115 had knowledge of the stimulus.

FIG. 3 is a flow chart illustrating the event related potential (ERP) subroutine 220 that measures the event related potential for the cerebral indicator signals received from the subject 115. The ERP subroutine 220 begins at step 310. In this step, the ERP subroutine 220 receives the cerebral indicator signals from the subject 115. Though not shown, the subject 115 produces cerebral indicator signals in response to processing the sensory signals described with reference to the sensory transmitter (see FIG. 1A). Step 310 is followed by step 315. In this step, the ERP subroutine 220 digitizes the received signals, which creates a series “O”. This digitizing may be measured with any type of conventional digitizer, such as a 250 Hz digitizer, a 400 Hz digitizer, or the like.

After digitizing the signal, the ERP subroutine 220 manipulates the digitized signal as shown in FIG. 3. Step 315 is followed by step 320. In this step, the ERP subroutine 220 runs the Fast Fourier transform on the original “O” data series received from the subject 115, which converts the digitized signals into individual frequency components. Step 320 is followed by step 325. In this step, the ERP subroutine 220 finds the peak frequency within a defined frequency range, such as the 40-90 Hz range. While other frequency ranges can be used, knowledge detection based on the use of an EEG gamma band frequency of 40 to 90 Hz or 30 to 105 Hz is most commonly used. Step 325 is followed by step 330. In this step, the ERP subroutine 220 finds the number of data points in the peak gamma frequency, which depends on the digitizing speed. In other words, this subroutine determines the number of data points within a simusoid corresponding to the peak frequency found in step 325.

Step 330 is followed by step 335. In this step, the ERP subroutine 220 smoothes data in defined regions in a manner that creates an “O-Gamma” series. More specifically, the ERP subroutine 220 applies successively running window averages of window lengths P−3, P−2, P, P+1, P+2, P+3 where P is the peak gamma frequency selected in step 330. When this window is iteratively run through the “O” series, this window will eliminate the sinusoids of the peak gamma frequency and the frequencies around it, leaving all of the other frequencies in place. In essence, this elimination occurs because the mean value of a window containing a sine wave is zero. When the first number in the window is replaced, the window is moved one window length to the right and the sine wave in this window, though at a different phase, still sums to zero.

Step 335 is followed by step 340. In this step, the ERP subroutine 220 defines, a series “Gamma” as the difference between the original series “O” and the series “O-Gamma”. Step 340 is followed by the end step 345. As the completed subroutine 220 ends, the knowledge determination algorithm 200 completes steps 220 and begins step 225 (see FIG. 2).

Turning now to FIG. 4, this figure is a flow chart illustrating the PD2i subroutine 225, which begins at step 410. In step 410, PD2i subroutine 225 receives electrophysiological data. While this is shown as a separate step, this data corresponds to the cerebral indicator signals received from the subject 140. Step 410 is followed by step 415. In step 415, the PD2i subroutine 225 calculates the vector difference lengths. More specifically, the PD2i subroutine 225 calculates the vector difference lengths, finds their absolute values, and then rank orders them. A single vector difference length is made between a reference vector that stays fixed at a point i and any one of all other possible vectors, j, in the data series. Each vector is made by plotting, in a multidimensional space called an embedding dimension, m. The coordinates of this dimension are defined by the values of m, which are in actuality the number of successive data points at each data point in the “Gamma” data series defined in block 340 (see FIG. 3). That is, a short segment of the gamma-enriched data is used to form the coordinates that make an m-dimensional vector. For example, 3 data points make a 3-dimensional vector (m=3), and 12 data points make a 12-dimensional vector (m=12). After calculating the reference vector, starting at a data-point i (i.e., i-vector), and the j-vector (one of any other vectors that can be made, starting at point j in the same data series), then the vector difference is calculated and its absolute value is stored in an array. All j-vectors are then made with respect to the single fixed i-vector. Then point-i is incremented and again all i-j vector difference lengths are determined. Then m is incremented and the whole i-j vector difference lengths are again calculated. These steps illustrate how the PD2i subroutine 225 completes step 420.

Step 420 is followed by step 425. In this step, the PD2i subroutine 225 calculates the correlation integrals for each embedding dimension (e.g., m, for point-i in the enriched gamma data series), where the fixed reference vector, or i-vector, is located. These correlation integrals generally indicate the degrees of freedom at a particular point in time, depending upon the slope in the scaling interval. Step 425 is followed by step 430 where the PD2i subroutine 225 uses the correlation integral determined in step 425. Then this subroutine restricts the scaling region to the initial small-end of the linear part of the correlation integral that lies above the unstable region caused by error resulting from the limitation on the speed of the digitizer. More specifically, this subroutine defines a correlation integral scaling region based on the plot length criterion. This criterion essentially restricts the scaling to the small log-R end of the correlation integral with the resulting property of insensitivity to data stationarity.

Step 430 is followed by the decision step 435. In this step, PD2i subroutine 225 determines whether the linearity criterion is satisfied. The linearity criterion evaluates the scaling region, which should be essentially linear. If the linearity criterion is satisfied, the “yes” branch is followed from step 435 to step 440. In step 440, the PD2i subroutine 225 determines whether the minimum scaling criterion is satisfied, which essentially means that there are a suitable number of data points within the scaling region. If the minimum scaling criterion is not satisfied, the PD2i subroutine 225 follows the “no” branch from step 435 to step 445. Step 445 also follows step 440 if the linearity criterion is not satisfied. In step 445, the PD2i subroutine 225 stores the mean, or average, slope and standard deviation as a −1.

When the minimum scaling criterion is satisfied, the “yes” branch is followed from step 440 to step 450. In step 450, the PD2i subroutine 225 stores the mean slope and standard deviation of the scaling region slopes of the correlation integrals for the convergent embedding dimensions. That is, the values are for the slopes where increasing m does not lead to a change in the slope of the scaling region for the associated point embedding dimension with the reference vector being at point i.

Step 455 follows both step 445 and step 450. In step 455, the PD2i subroutine 225 selects the next PD2i point, which has either an incremented i or an incremented m coordinate if all i-coordinates at that value of m have been used. Step 455 is followed by step 460. In this step, the PD2i subroutine 225 determines whether all the PD2i points and m s are selected. If there are remaining unselected values, the “no” branch is followed from step 460 to step 415, which essentially repeats the subroutine 225.

If it is determined that all are selected at step 460, the “yes” branch is followed from step 460 to step 465. In step 465, the PD2i subroutine 225 determines whether the convergence criterion is satisfied. Essentially, this criterion analyzes the convergent PD2i slope values and determines if they converged more than a predetermined amount. If the convergence criterion is satisfied, step 465 is followed by step 470 (i.e., follow the yes” branch). In this step, the PD2i subroutine 225 displays, “Accepted.” If it is determined that the convergence criterion is not satisfied, the “no” branch is followed from step 465 to step 475. In step 475, the PD2i subroutine 225 displays, “Not Accepted.” In other words, “Not Accepted” indicates that the PD2i is invalid for some reason, such as noise. The end step 480 follows both step 470 and step 475, which causes the PD2i subroutine 225 to end. As this subroutine ends, the knowledge determination algorithm 200 completes step 225 and begins step 230 (see FIG. 2).

Description of the System's Applications

The knowledge determination system 100 creates substantial advantages over conventional knowledge determination methods, which facilitates its application in a host of scenarios. For example, this system can be used as a lie detection device. The sensory signal transmitted may be an audible voice that states an individual's name. Because the knowledge determination system 100 detects involuntary subconscious responses inherent to a subject's brain, this system has considerably greater accuracy than responses solely based on the autonomic nervous system (e.g., heartbeats, respirations, galvanic skin responses, and the like). Alternatively, the novel knowledge determination system 100 can be incorporated within an airport security system at the security checkpoint. To facilitate this use, the sensory transmitter 105 and the detector 107 may be located within a scanner that people walk through. In addition, the knowledge determination system 100 can be used in aiding and treating medical conditions, such as evaluating patients in denial. One skilled in the art will appreciate that the brain's frontal lobe is involved in many psychiatric disorders, which is involved in the brain's analysis of the sensory input that the knowledge determination system 100 measures. Finally, a neurologist can use the knowledge determination system 100 on patients suffering from a lucid coma, where there is no brain damage but they cannot respond. These patients will show a reduced PD2i direction indicative that meaning is associated based on prior knowledge of the presented stimulus.

The particular embodiments disclosed above are illustrative only, as the knowledge determination system 100 may be modified and practiced in different, but equivalent, manners apparent to those skilled in the art having the benefit of the teachings herein. Furthermore, no limitations are intended to the details of construction or design herein shown, other than as described in the claims below. It is therefore evident that the particular embodiments disclosed above may be modified and all such variations are considered within the scope and spirit of the invention. Accordingly, the protection sought herein is set forth in the claims below.

The invention claims:

1. A method for determining whether a subject has knowledge of a stimulus, the steps of the method comprising:
generating a sensory signal corresponding to the stimulus for receipt by the subject;
collecting a cerebral indicator signal involuntarily generated in response to the subject processing the sensory signal;
identifying whether degrees of freedom in the cerebral indicator signal either increased or decreased;
determining whether the subject has knowledge of the stimulus depending on whether the degrees of freedom increased or decreased; and
associating knowledge of the stimulus with the subject if it is determined that the subject has knowledge of the stimulus.

2. The method of claim 1, wherein identifying whether the degrees of freedom in the cerebral indicator signal either increased or decreased, further comprises determining an event-related potential.

3. The method of claim 2, wherein determining an event-related potential further comprises steps of:
digitizing the cerebral indicator signal to generate a first data set;
converting the first data set into individual frequency components;
identifying a peak frequency within a frequency range associated with the individual frequency components; and
determining a number of data points associated with a second data set, wherein the second data set is associated with the peak frequency.

4. The method of claim 3, further comprising the steps of:
creating a third data set by smoothing the second data set; and
defining a fourth data set as the difference between the third data set and the first data set.

5. The method of claim 3, wherein identifying whether the degrees of freedom in the cerebral indicator signal either increased or decreased, further comprises the step of determining a PD2i.

6. The method of claim 5, wherein determining a PD2i further comprises the steps of:
calculating vector difference lengths associated with the cerebral indicator signal;
storing the vector difference lengths in an array associated with an embedding dimension;
calculating correlation integrals for the embedding dimension; and
defining a scaling region associated with the correlation integral.

7. The method of claim 6, wherein determining a PD2i further comprises the steps of:
determining whether a linearity criterion is satisfied;
determining whether a minimum scaling criterion is satisfied;
storing a mean slope and deviation; and
determining whether a convergence criterion is satisfied.

8. The method of claim 7, wherein determining a PD2i further comprises the step of displaying a notice in response to determining whether a convergence criterion is satisfied.

9. The method of claim 2, wherein determining whether the subject has knowledge of the stimulus further comprises the step of determining a difference of PD2i values.

10. A knowledge determination system for determining whether a subject has knowledge of a stimulus comprising:
a sensory transmitter that sends a sensory signal corresponding to the stimulus for receipt by the subject;
a detector positioned to receive an involuntary subconscious cerebral indicator signal in response to the subject processing the sensory signal, wherein the detector produces a detected signal after processing the involuntary subconscious cerebral indicator signal; and
a processor coupled to receive the detected signal and associate knowledge of the stimulus with the subject.

11. The knowledge determination system of claim 10, wherein the processor farther comprises software that associates knowledge of the stimulus with the subject, by executing the steps of:
generating a sensory signal corresponding to the stimulus, for receipt by the subject;
collecting a cerebral indicator signal involuntarily generated in response to the subject processing the sensory signal;
identifying whether degrees of freedom in the involuntary subconscious cerebral indicator signal either increased or decreased;
determining whether the subject has knowledge of the stimulus depending on whether the degrees of freedom increased or decreased; and associating knowledge of the stimulus with the subject if it is determined that the subject has knowledge of the stimulus.

12. The knowledge determination system of claim 10, wherein the sensory signal is selected from the group of signals consisting of audible signals and visual signals.

13. The knowledge determination system of claim 10, wherein the detector is selected from the group of detectors consisting of magneto encephalograph and an electronencephalogram.

14. The knowledge determination system of claim 10, wherein the processor is a computer.

15. The knowledge determination system of claim 10, wherein the knowledge determination system is included with a device selected from the group consisting of an airport security system and a lie detection device.

16. The knowledge determination system of claim 10, wherein the subject has a medical condition selected from the group consisting of a psychiatric disorder, a lucid coma, and denial.

17. A computer-readable medium for determining whether a subject has knowledge of a stimulus, comprising the steps:
generating a sensory signal corresponding to the stimulus for receipt by the subject;
collecting a cerebral indicator signal involuntarily generated in response to the subject processing the sensory signal;
identifying whether degrees of freedom in the cerebral indicator signal of the subject either increased or decreased;
determining whether the subject has knowledge of the stimulus depending on whether the degrees of freedom increased or decreased; and
associating knowledge of the stimulus with the subject if it is determined that the subject has knowledge of the stimulus.

18. The computer readable medium of claim 17, wherein identifying whether the degrees of freedom in the cerebral indicator signal either increased or decreased, further comprises the steps of:
digitizing the cerebral indicator signal to generate a first data set;
converting the first data set into individual frequency components;
identifying a peak frequency within a frequency range associated with the individual frequency components;
determining a number of data points associated with a second data set, wherein the second data set is associated with the peak frequency;
creating a third data set by smoothing the second data set; and
defining a fourth data set as the difference between the third data set and the first data set.

19. The computer readable medium of claim 17, wherein identifying whether the degrees of freedom in the cerebral indicator signal either increased or decreased, further comprises the step of determining a PD2i.

20. The method of claim 19, wherein determining a PD2i further comprises the steps of:
calculating vector difference lengths associated with the cerebral indicator signal;
storing the vector difference lengths in an array associated with an embedding dimension;
calculating correlation integrals for the embedding dimension;
defining a scaling region associated with the correlation integral;
determining whether a linearity criterion is satisfied;
determining whether a minimum scaling criterion is satisfied;
storing a mean slope and deviation; and
determining whether a convergence criterion is satisfied.
